(19) United States
(12) Patent Application Publication
(10) Pub. No.: US 2023/0107159 A1
(43) Pub. Date: Apr. 6, 2023

(54) METHOD FOR CATALYTICALLY PRODUCING AN ALKYL FORMATE

(71) Applicant: OXFA GMBH, SCHESSLITZ (DE)

(72) Inventors: Jakob Albert, Hamburg (DE); Chiraphat KUMPIDET, Pathum Thani (TH)

(21) Appl. No.: 17/795,380

(22) PCT Filed: Jan. 26, 2021
(86) PCT No.: PCT/EP2021/051717

§ 371 Date: Jul. 26, 2022

(30) Foreign Application Priority Data
Jan. 27, 2020 (EP) 20153906.1

(51) Int. Cl. C07C 67/40; C07C 51/235; B01J 23/00

(52) U.S. Cl. CPC C07C 67/40; C07C 51/235; B01J 23/002; B01J 2523/00

(57) ABSTRACT

The invention relates to a method for catalytically producing an alkyl formate, wherein at least one alpha-hydroxy aldehyde, at least one alpha-hydroxy carboxylic acid, at least one carbohydrate, and/or at least one glycoside is reacted by means of a vanadium-oxygen compound, which contains vanadium in the oxidation stage +IV or +V, or a salt thereof as a catalyst in the solution, wherein the solution contains an alkanol, and the alkyl formate produced as a reaction product is separated from at least one other resulting reaction product. The catalyst which is reduced during the catalytic reaction is restored to its starting state in an oxidation process.

The invention relates to a process for catalytic production of an alkyl formate.

In the process a polyoxometalate ion serving as catalyst of general formula [PMoₓV_{y}O₄₀]ⁿ⁻ may be reacted in a solution with an alpha-hydroxyaldehyde, an alpha-hydroxycarboxylic acid, a carbohydrate or a glycoside. Therein 6≤x≤11, 1≤y≤6 and x+y=12, where n, x and y are each an integer and 3<n<10 is possible.

A process in which such a catalyst is reacted with an alpha-hydroxyaldehyde, an alpha-hydroxycarboxylic acid, a carbohydrate or a glycoside is known from WO 2016/120169 A1. The process known therefrom is used to produce formic acid.

US 2005/0154226 A1 discloses a process for oxidation of a gaseous charge comprising methanol and/or dimethyl ether to produce a product containing primarily dimethoxymethane or primarily methyl formate. The charge is contacted with an oxygen-containing gas and a supported heteropolyacid Keggin catalyst containing molybdenum or molybdenum and vanadium. No homogeneous methanol reactions were observed under the conditions specified in the exemplary embodiments.

Albert, Jakob, et al., Energy and Environmental Science 5 (2012), pages 7956 to 7962 discloses a selective oxidation of biomass into formic acid using the polyoxometalate H₅PV₂Mo₁₀O₄₀ as a homogeneous catalyst, oxygen as an oxidant, water as a solvent and p-toluenesulfonic acid as an additive. The described oxidation is carried out at 90° C. and an oxygen partial pressure of 30 bar. A yield of up to 53% formic acid after 24 hours is reported.

Tang, Z. et al., ChemSusChem 2014, 7, pages 1557 to 1567 discloses a vandyl cation-catalyzed conversion of cellulose into formic acid and lactic acid. It especially discloses the use of VOSO₄ as a catalyst for conversion of glucose into formic acid and into lactic acid. The formation of CO₂ in this reaction restricts the yield of formic acid to just above 50%. However, it was found that addition of methanol or ethanol to the reaction system inhibits the formation of CO₂ during conversion of glucose under aerobic conditions, thus allowing the yield of formic acid to be increased to 70% to 75%.

EP 2 922 815 B1 discloses a process for catalytic production of methyl formate by reaction of methanol with carbon monoxide in the presence of a catalyst system containing alkali metal formate and alkali metal alkoxide.

It is an object of the present invention to provide an alternative process for producing alkyl formate.

The object is achieved by the features of claim 1. Advantageous embodiments are apparent from the features of claims 2 to 15.

Provided according to the invention is a process for catalytic production of alkyl formate, wherein at least one alpha-hydroxyaldehyde, at least one alpha-hydroxycarboxylic acid, at least one carbohydrate and/or at least one glycoside as substrate is reacted in a solution using a vanadium-oxygen compound containing vanadium in the oxidation state +IV or +V or a salt thereof as catalyst, wherein the solution contains an alkanol. The alkyl formate formed as a reaction product is separated from at least one further reaction product formed, for example dimethoxyalkane, in particular dimethoxymethane. The catalyst reduced during the catalytic reaction is returned to its starting state by oxidation. The separating of the at least one further reaction product may be effected using known measures, such as extraction or evaporation and/or by distillation. Either the alkyl formate or the further reaction product may be isolated from the solution.

The catalyst is a polyoxometalate ion of general formula [PMoₓV_{y}O₄₀]ⁿ⁻, wherein 6≤x≤11, 1≤y≤6 and x+y=12, [WₓV_{y}O₁₉]ⁿ⁻, wherein x+y=6, 3≤x≤5 and 1≤y≤3 or [P₂WₓV_{y}O₆₂]ⁿ⁻, wherein x+y=18, 12≤x≤17 and 1≤y≤6, or a VO²⁺-containing salt, in particular VOSO₄, or a [VO₃]⁻-containing salt, in particular NH₄VO₃, wherein n, x and y are in each case an integer. The value of n results from the partial charges of the elements present in the catalyst. In [PMoₓV_{y}O₄₀]ⁿ⁻ for example, 3<n<10. The polyoxometalate ion [PMoₓV_{y}O₄₀]ⁿ⁻, in particular [PMo₇V₅O₄₀]⁸⁻ (HPA-5), has proven suitable. Due to the specific structures formed by the ions [PMoₓV_{y}O₄₀]ⁿ⁻ is also known as the Keggin ion, [WₓV_{y}O₁₉]ⁿ⁻ as the Lindqvist ion and [P₂WₓV_{y}O₆₂]ⁿ⁻ as the Wells-Dawson ion.

The substrate may be an alpha-hydroxyaldehyde, an alpha-hydroxycarboxylic acid, a carbohydrate or a glycoside or any desired mixture of in each case one or more alpha-hydroxyaldehydes, one or more alpha-hydroxycarboxylic acids, one or more carbohydrates and/or one or more glycosides.

In the catalytic production of the alkyl formate from the carbohydrate or the glycoside the oxidative cleavage of adjacent carbon atoms of the carbohydrate or of the sugar constitutent of the glycoside, wherein an OH group is bonded to at least one of these carbon atoms, always forms an alpha-hydroxyaldehyde or an alpha-hydroxycarboxylic acid as an intermediate, Therefore, the alpha-hydroxyaldehyde and the alpha-hydroxycarboxylic acid may each also be directly employed as the substrate.

The inventors have found not only that the abovementioned catalyst and the abovementioned substrate result in formation of alkyl formate but also that the presence of an alkanol in the solution ensures that total oxidation of parts of the substrate to form CO₂ and H₂O is reduced or even completely prevented according to the alcohol proportion. In addition to the alkanol the solution may contain at least one further solvent miscible therewith. The further solvent may be water. However, the solution may also contain only the alkanol or a mixture of alkanols as the sole solvent.

When the solution also contains water in addition to the alkanol, formic acid is formed as a further reaction product in addition to the alkyl formate. The yield of formic acid is markedly higher in the presence of the alkanol than in purely aqueous solution.

The alkanol may be an unbranched alkanol, i.e. an n-alkanol, and/or comprise 1 to 4 carbon atoms. The alkanol may be methanol, ethanol, n-propanol or n-butanol. In particular, the alkanol may be methanol. Methanol is advantageous on account of the methyl formate formed therefrom, since methyl formate has a very low boiling point of only 32° C. It may therefore be easily isolated by evaporation/obtained by distillation from the mixture of formic acid and methyl formate formed in the solution in the presence of water without auxiliaries and without formation of an azeotrope. However, ethanol, n-propanol oder n-butanol do not have the toxicity of methanol and and are easier to handle on account of the reduced safety measures resulting therefrom. The alkyl formates ethyl formate and n-propyl formate resulting from ethanol and n-propanol likewise have boiling points below the boiling point of formic acid of 101° C. These alkyl formates too may be isolated by evaporation/distillation from the mixture of formic acid and alkyl formate formed in the solution in the presence of water.

The isolating of the resulting alkyl formate from the solution has the result that the process also allows advantageous and highly selective production of formic acid with a markedly higher yield than in a reaction of the respective substrate in the absence of an alkanol. Selectivities in respect of the formation of alkyl formate and/or formic acid of more than 90% were achieved. This was possible even without the use of an additional reaction accelerator or extractant. The inventors have recognized that the higher the proportion of alkanol in the solution, the higher the proportion of alkyl formate produced in the reaction, When the solvent consists exclusively of the alkanol no formic acid is formed in the reaction of the substrate. Conversely the proportion of formic acid produced in the reaction may be elevated by increasing the water content in the solution. However, the increased selectivity of the reaction in respect of the formation of the alkyl formate and formic acid and the reduced formation of CO₂ in the reaction is detectable even at a low amount of alkanol in the, especially water-containing, solution.

In one embodiment of the process the solution contains at least 5% by weight of alkanol, in particular at least 10% by weight of alcohol, in particular at least 20% by weight of alkanol, in particular at least 30% by weight of alkanol, in particular at least 50% by weight of alkanol, in particular at least 70% by weight of alkanol, in particular at least 80% by weight of alkanol, in particular at least 90% by weight of alkanol, in particular at least 95% by weight of alkanol, in particular 100% by weight of alkanol.

In addition to the alkanol the solution may contain at least one further nonaqueous solvent. The presence of the further nonaqueous solvent instead of water also increases the recited selectivity and reduces the formation of CO₂.

In one embodiment of the process the reduced catalyst is returned to its starting state by oxidation using oxygen or an oxygen-containing oxidant. Oxidation using oxygen may be an oxidation using molecular oxygen as pure gas or in a gas mixture containing the molecular oxygen, for example air or synthetic air. Synthetic air is generally a gas mixture consisting of oxygen and nitrogen in which the oxygen proportion is in the range from 19.5% by volume to 21.5% by volume.

The oxygen-containing oxidant may be a peroxide, in particular H₂O₂, or N₂O.

Oxidation using molecular oxygen may be carried out—in the case of oxygen as pure gas—at an oxygen pressure or—in the case of a gas mixture—at an oxygen partial pressure in the range from 1 bar to 250 bar, in particular 1 bar to 120 bar, in particular 1 bar to 80 bar, in particular 1 bar to 50 bar, in particular 1 bar to 30 bar, in particular 5 bar to 20 bar, in particular 5 bar to 10 bar. To effect oxidation the solution may be subjected to molecular oxygen for example in a static mixer or by vigorous stirring. It has proven advantageous and efficient for the process when the conversion of the at least one alpha-hydroxyaldehyde, the at least one alpha-hydroxycarboxylic acid, the at least one carbohydrate and/or the at least one glucoside using the catalyst is carried out at a temperature of not more than 150° C., in particular not more than 120° C., in particular in the range from 65° C. to 120° C., in particular in the range from 70° C. to 100° C., in particular in the range from 70° C. to 90° C.

The alpha-hydroxycarboxylic acid may be glycolic acid or lactic acid and the carbohydrate may be a monosaccharide, in particular having 5 or 6 carbon atoms, a disaccharide, in particular having 12 carbon atoms, an oligosaccharide or a polysaccharide. The monosaccharide may be an aldose, in particular glucose or xylose. The disaccharide may be sucrose or cellobiose. The oligosaccharide may be a heterooligosaccharide. The polysaccharide may be starch, cellulose, hemicellulose or a heteropolysaccharide, in particular a xylan.

In one working example the at least one alpha-hydroxyaldehyde, the at least one alpha-hydroxycarboxylic acid, the at least one carbohydrate and/or the at least one glycoside are present in an, especially renewable, raw material or a residue derived from a conversion of the raw material. The raw material may be a material of biological origin, in particular vegetable origin. The raw material may be lignocellulose-containing biomass, for example woody plant material or sawdust. The raw material may be an untreated, i.e, chemically undigested, raw material. Chemical digestion can cause catalyst-inactivating chemicals to be introduced into the raw material. The residue or the renewable raw material may be a plant, a fungus or bacteria or constituents of plants, fungi or bacteria, wood, in particular in the form of wood flour or wood shavings, paper, in particular waste paper, algae, cyanobacteria or silage. The alpha-hydroxyaldehyde, the alpha-hydroxycarboxylic acid, the carbohydrate or the glycoside may also comprise a mixture of at least two of the recited substances or be formed from at least one of the recited substances or the mixture, such as is the case for lignite or peat.

The invention will hereinbelow be more particularly elucidated with reference to working examples.

In a first working example 1 mmol of glucose was dissolved in each case in 10 g of solvent consisting of water and/or methanol in varying weight fractions and 0.1 mmol of the polyoxometalate ion [PMo₇V₅O_{40]}⁸⁻ (=HPA-5) was added. This solution was stirred for 24 h at 1000 revolutions per minute while being held at a temperature of 90° C. and subjected to oxygen at an oxygen partial pressure of 20 bar. The results are summarized in the following table 1:

TABLE 1

| | w_{H}_{₂}_{O,before}/ | w_{H}_{₂}_{O,after}/ | X_{Glu}/% | | | | | FA:MF | |
|---|---|---|---|---|---|---|---|---|---|
| Substrate | % by wt. | % by wt. | (HPLC) | DME | FAI | MM | DMM | ratio | Y_{CO}_{₂}_{/CO}/% |
| Glucose | 0 | 4.4 | 100 | — | — | — | x | 0:100 | —/— |
| Glucose | 10 | 14.0 | 100 | — | — | x | x | 20:80 | 0.5/— |
| Glucose | 20 | | 99 | — | — | x | x | 25:75 | 0.6/— |
| Glucose | 30 | | 99 | — | — | x | x | 35:65 | 1.0/0.2 |
| Glucose | 40 | | 98 | — | — | x | x | 42:58 | 1.2/0.2 |
| Glucose | 48 | | 98 | — | — | x | x | 44:56 | 1.8/0.3 |
| Glucose | 49 | | 98 | — | — | x | x | 46:54 | 1.6/0.2 |
| Glucose | 70 | | 97 | — | — | x | x | 61:39 | 2.2/0.2 |
| Glucose | 90 | | 93 | — | — | x | x | 81:19 | 3.3/0.3 |
| Glucose | 100 | | 100 | — | — | — | — | 100:0 | 27.4/0.4 |

The column “w_{H}_{₂}_{O,before}/% by wt.” indicates the percentage by weight of water in the solution before the reaction. If this proportion is 0% by weight the solution consists exclusively of methanol. If this proportion is 100% by weight the solution contains no methanol. The column “w_{H}_{₂}_{O,after}/% by wt.” indicates the percentage by weight of water in the reaction solution after the reaction. “X_{Glu}/% (HPLC)” indicates the percentage conversion of the employed glucose determined by high performance liquid chromatography (HPLC). In the above table 1 and subsequent tables the abbreviations are defined as follows:
DME: Dimethyl ether
FAI: Formaldehyde
MM: Methoxymethanol
DMM: Dimethoxyrnethane
FA: Formic acid
ME: Methyl formate

The presence of the reaction products DME, FAI, MM and DMM was determined by ¹³C-NMR spectroscopy. The presence of FA and MF was likewise determined by ¹³C-NMR spectroscopy and the quantity ratios of formic acid to methyl formate indicated in the column “FA:MF” by determining the ratio of the peak areas for formic acid and methyl formate in the ¹³C-NMR spectrum. The column “Y_{CO}_{₂}_{/CO}/%” indicates the percentage yields of CO₂ und CO determined by gas chromatography and based on the glucose employed in each case. If no reaction product was detected this was indicated with an “-”, otherwise with an “x”,

It is apparent from table 1 that at an initial water content of 0% by weight, i.e. 100% by weight methanol, no CO₂, no CO and no formic acid has been formed and accordingly the FA:ME ratio is 0:100. For all reactions in which the solvent contained methanol dimethoxymethane was formed as a byproduct. Table 1 also shows that the higher the proportion of methanol in the solution, the higher the proportion of methyl formate formed. Conversely, the higher the proportion of water in the solution, the higher the proportion of formic acid formed. In the absence of methanol only formic acid, CO₂ and CO were formed. Methyl formate was not formed. Table 1 also shows in the column “Y_{CO}_{₂}_{/CO}/%” that in the absence of methanol a relatively large proportion of CO₂ is formed but even 10% methanol is sufficient to markedly reduce CO₂ and CO formation. The table also shows that only the water content present at commencement of the reaction is decisive for the reaction products. The water formed during the reaction of 100% methanol does not bring about formation of formic acid.

In a second working example 1 mmol of glucose was dissolved in each case in 10 g of methanol as solvent and 0.5 mmol of vanadium present in a catalyst was added. The catalysts employed were the polyoxometalate ion [PMo₇V₅O₄₀]⁸⁻ (=HPA-5), VOSO₄, NH₄VO₃ and K₅V₃W₃O₁₉. This solution was stirred for 24 hours at 1000 revolutions per minute while being held at a temperature of 90° C. and subjected to oxygen at an oxygen partial pressure of 20 bar. The results are summarized in the following table 2.

TABLE 2

| | | w_{H}_{₂}_{O,after}/ | | | | | | | |
|---|---|---|---|---|---|---|---|---|---|
| Substrate | Catalyst | % by wt. | DME | FAI | MM | DIMM | FA | MF | Y_{CO}_{₂}_{/CO}/% |
| None | HPA-5 | 2.1 | x | — | — | x | — | — | —/— |
| Glucose | HPA-5 | 4.4 | — | — | (x) | x | — | x | —/— |
| Glucose | VOSO₄ | 4.2 | — | — | (x) | x | — | x | 0.6/— |
| Glucose | NH₄VO₃ | 2.9 | — | — | x | (x) | — | x | 1.8/0.2 |
| Glucose | K₅V₃W₃O₁₉ | 2.1 | — | — | x | (x) | — | x | 1.3/0.2 |

The column “w_{H}_{₂}_{O,after}/% by wt.” indicates the percentage by weight of water in the reaction solution after the reaction. The column “Y_{CO}_{₂}_{/CO}/%” indicates the percentage yields of CO₂ and CO determined by gas chromatography and based on the glucose employed in each case. If no reaction product was detected this was indicated with a “-”, otherwise with an “x”, “(x)” indicates that only traces of the respective reaction product were detected.

Table 2 shows that various vanadium-oxygen compounds containing vanadium in the +IV or +V oxidation state or salts thereof may be employed as catalyst for producing the alkyl formate. Table 2 further confirms that initial absence of water has the result that no formic acid is formed, even if water is formed in the course of the reaction.

In a third working example beech shavings were suspended or 68% by weight of molasses were dissolved in 100 g of methanol as solvent and 1 mmol of the polyoxometalate ion [PMo₇V₅O₄₀]⁸⁻ (=HPA-5) added as catalyst. This suspension/solution was stirred at 1000 revolutions per minute for 24 hours while being held at a temperature of 90° C. and subjected to oxygen at an oxygen partial pressure of 20 bar. The results are summarized in the following table 3.

TABLE 3

| | w_{H}_{₂}_{O,substrate}/ | w_{H}_{₂}_{O,after}/ | | | | | | | |
|---|---|---|---|---|---|---|---|---|---|
| Substrate | % by wt. | % by wt. | DME | FAI | MM | DMM | FA | MF | Y_{CO}_{₂}_{/CO}/% |
| Beech | | 2.7 | x | — | x | x | — | x | —/— |
| shavings | | | | | | | | | |
| Molasses | 31.8 | 2.0 | x | — | — | x | — | x | 3.6/— |
| (68% by wt.) | w_{H}_{₂}_{O,before} = | | | | | | | | |
| | 0.13 | | | | | | | | |

The column “W_{H}_{₂}_{O,substrate}% by wt.” indicates the percentage by weight of water in the substrate. The column “w_{H}_{₂}_{O,before}/% by wt.” indicates the percentage by weight of water in the solution before the reaction. The column “w_{H}_{₂}_{O,after}/% by wt.” indicates the percentage by weight of water in the reaction solution after the reaction. If no reaction product was detected this was indicated with a “-”, otherwise with an “x”. Before the reaction an organic (CHNS) elemental analysis was performed on each of the employed substrates to determine in each case for each of the substrates the percentage by weight of carbon, hydrogen, nitrogen and sulfur and from the difference from 100% the percentage by weight of oxygen. Weighted with the molar masses this gives the respective amount of substance fraction, on the basis of which the percentage yield of CO₂ indicated in the column “Y_{CO}_{₂}_{/CO}/%” was determined. A formation of CO could not be detected for any of the substrates. For beech shavings as the substrate no CO₂ formation could be detected either.

Table 3 shows that substrates other than glucose may also be employed. Not shown here but also detected by the inventors for these substrates was the formation of formic acid as soon as the solution also contained water at commencement of the reaction in addition to the alkanol.

In a fourth working example in each case 1 mmol of glucose, 1 mmol of xylose, 1 mmol of lactic acid, 1 mmol of glyceraldehyde, 1 mmol of glycolaldehyde or 1 mmol of erythrose were dissolved in 10 g of methanol as solvent. The resulting solutions were in each case admixed with 0.1 mmol of HPA-5 as catalyst. These solutions were stirred for 24 hours at 1000 revolutions per minute, held at a temperature of 90° C. and subjected to oxygen at an oxygen partial pressure of 20 bar. The results are summarized in the following table 4:

TABLE 4

| Substrate | MF + FA | MF | FA | Glyoxal | Erythrose | Glycolaldehyde | Y_{CO}_{₂}_{/CO}/% |
|---|---|---|---|---|---|---|---|
| Glucose | 95.7 | 44 | 51.7 | 0.3 | 1.7 | 1.5 | 0.7/0.1 |
| Xylose | 95.7 | 44 | 51.7 | — | 2.1 | 1.6 | 0.5/0.1 |
| Lactic acid | 82 | 37.7 | 44.3 | — | — | 1 | 17/— |
| Glyceraldehyde | 99 | 45.5 | 53.5 | — | — | — | 1/— |
| Glycolaldehyde | 57.5 | 26.5 | 31.1 | — | — | 41.4 | 0.6/0.5 |
| Erythrose | 98 | 45.1 | 52.9 | — | — | 0.9 | 1/0.1 |

The numerical values in the table in each case indicate the percentage proportion of the reaction product based on the altogether obtained reaction products. The presence of the reaction products glyoxal, erythrose, glycolaldehyde, MF and FA and the percentage proportions thereof were determined by ¹³C-NMR spectroscopy. The column “Y_{CO}_{₂}_{/CO}/%” indicates the percentage yields of CO₂ and CO determined by gas chromatography and based on the substrates employed in each case. If no reaction product was detected this was indicated with a “-”. Table 4 shows that other substrates may also be used for producing the alkyl formate in addition to glucose.

1. A process for catalytic production of an alkyl formate, wherein at least one alpha-hydroxyaldehyde, at least one alpha-hydroxycarboxylic acid, at least one carbohydrate and/or at least one glycoside is reacted in a solution using a vanadium-oxygen compound containing vanadium in the oxidation state +IV or +V or a salt thereof as catalyst, wherein the solution contains an alkanol, wherein the alkyl formate formed as a reaction product is separated from at least one further reaction product formed, wherein the catalyst reduced during the catalytic reaction is returned to its starting state by oxidation, wherein the catalyst is a polyoxometalate ion of general formula [PMoₓV_{y}O₄₀]ⁿ, wherein 6≤x≤11, 1≤y≤6 and x+y=12, [WₓV_{y}O₁₉]ⁿ⁻, wherein x+y=6, 3≤x≤5 and 1≤y≤3, or [P₂WₓV_{y}O₆₂]ⁿ⁻, wherein x+y=18, 12≤x≤17 and 1≤y≤6, or a VO²⁺-containing salt or a [VO_{3]}⁻-containing salt, wherein n, x and y are in each case an integer.

2. The process as claimed in claim 1, wherein the VO²⁺-containing salt is VOSO₄ and the [VO₃]⁻-containing salt is NH₄VO₃.

3. The process as claimed in claim 1, wherein the catalyst is [PMo₇V₅O₄₀]⁸⁻.

4. The process as claimed in claim 1, wherein the alkanol is an n-alkanol and/or comprises 1 to 4 carbon atoms.

5. The process as claimed in claim 1, wherein the alkanol is methanol, ethanol, n-propanol or n-butanol, in particular methanol.

6. The process as claimed in claim 1, wherein the solution also contains water in addition to the alkanol with the result that formic acid is formed as a further reaction product in addition to the alkyl formate.

7. The process as claimed in claim 1, wherein the solution contains at least 10% by weight of alkanol, in particular at least 50% by weight of alkanol.

8. The process as claimed in claim 1, wherein the solution contains at least 90% by weight of alkanol, in particular at least 95% by weight of alkanol.

9. The process as claimed in claim 1, wherein the reduced catalyst is returned to its starting state by oxidation using oxygen or an oxygen-containing oxidant.

10. The process as claimed in claim 9, wherein the oxidation using oxygen is an oxidation using molecular oxygen as pure gas or in a gas mixture containing the molecular oxygen, in particular air, and the oxygen-containing oxidant is a peroxide, in particular H₂O₂, or N₂O.

11. The process as claimed in claim 10, wherein the oxidation using molecular oxygen is performed at an oxygen pressure or oxygen partial pressure in the range from 1 bar to 50 bar, in particular 5 bar to 10 bar.

12. The process as claimed in claim 1, wherein the reacting of the at least one alpha-hydroxyaldehyde, the at least one alpha-hydroxycarboxylic acid, the at least one carbohydrate and/or the at least one glycoside using the catalyst is carried out at a temperature of not more than 150° C., in particular in a range from 70° C. to 100° C.

13. The process as claimed in claim 1, wherein the alpha-hydroxycarboxylic acid is glycolic acid or lactic acid and the carbohydrate is a monosaccharide, in particular having 5 or 6 carbon atoms, a disaccharide, in particular having 12 carbon atoms, an oligosaccharide or a polysaccharide.

14. The process as claimed in claim 13, wherein the monosaccharide is an aldose, in particular glucose or xylose, the disaccharide is sucrose or cellobiose, the oligosaccharide is a heterooligosaccharide and the polysaccharide is starch, cellulose, hemicellulose or a heteropolysaccharide, in particular a xylan.

15. The process as claimed in claim 1, wherein the at least one alpha-hydroxyaldehyde, the at least one alpha-hydroxycarboxylic acid, the at least one carbohydrate and/or the at least one glycoside is/are present in an, especially renewable, raw material or a residue derived from a conversion of the raw material.
